(19) United States
(12) Patent Application Publication
(10) Pub. No.: US 2004/0043947 A1
(43) Pub. Date: Mar. 4, 2004

(54) Methods of treating wounds or ulcers of the foot, ankle or lower extremity of the leg

(76) Inventor: Steven A. Gersh, Severna Park, MD (US)

Correspondence Address: ARMSTRONG, WESTERMAN & HATTORI, LLP, Suite 220, 502 Washington Avenue, Towson, MD, 21204, US

(21) Appl. No.: 10/231,873

(22) Filed: Aug. 29, 2002

(51) Int. Cl. A61K 31/704; A61K 31/573

(52) U.S. Cl. 514/39; 514/171

(57) ABSTRACT

The invention is directed to a topical composition containing neomycin, polymyxin and hydrocortisone for treating difficulty healing wounds or ulcers of the foot, ankle and lower extremity of the leg, and more particularly treating wounds and ulcers of diabetic patients.

### FIELD OF THE INVENTION

The herein disclosed invention finds applicability for treating difficultly healing wounds and ulcers of the foot and lower extremity of the leg, and more specifically, treating wounds and ulcers of the feet of diabetic patients.

### BACKGROUND OF THE INVENTION

Wounds of the foot and particularly diabetic lesions are difficult to treat and accordingly more effective therapies are always being sought.

The therapeutic composition of the herein disclosed invention has been used previously as an antibiotic anti-inflammatory for problems in the ear canal. The composition has also been used previously by podiatrists as a post-operative medication following ingrown toenail surgery where phenol is applied to the nail matrix or root. Phenol is a strong acid which is used to kill the nail matrix so the offending painful nail will not grow back.

The inventor who is a podiatrist has found that certain types of lesions which are slow healing and difficult to cure require a particular type of treatment. This is particularly true in diabetic patients and patients with slow healing wounds and ulcers of the foot. The inventor has found that lesions of the lower extremity and particularly diabetic lesions of the foot are difficult to cure, and heal with great difficulty. Before using the topical composition of this invention, the inventor was unsuccessful in using both over-the-counter and prescription topical medications such as saline and/or betadine wet or dry dressing, neosporin, bacitracin, triple antibiotic ointment, silvadene cream, bactroban ointment, santyl, regranex, amongst others.

While antibiotics have been used to treat infections and anti-inflammatory steroids have been used to treat inflammation, the mixture of antibiotics and steroids as envisioned by the invention has not been used to treat diabetic ulcers of the foot which are difficult to heal.

Prior Art References

Fischetti (U.S. Pat. No. 6,056,955) teaches a topical composition for application to dermal tissue to rid the tissue of infection. The composition may contain among other ingredients, anti-inflammatory and anti-microbial agents. Note that hydrocortisone is taught (col. 6, line 11); polymycin and neomycin are taught (col. 6, line 30).

Grollier (U.S. Pat. No. 6,136,332) teaches dermatologic compositions containing neomycin (col. 5, line 31), polymyxin (col. 5, line 35) and hydrocortisone (col. 5, line 34).

Osipow (U.S. Pat. No. 6,214,318) teaches topical compositions to be applied to the skin containing polymyxin, neomycin and hydrocortisone (col. 6, lines 44-46).

Sun (U.S. Pat. No. 6,231,875) teaches topical compositions for the treatment of human nail and skin diseases; neomycin is taught (col. 5, line 8), polymyxin is taught (col. 5, line 16) and hydrocortisone is taught (col. 5, line 25).

Physician's Desk Reference, 2002, page 2246, teaches ointments or creams containing neomycin, polymyxin and cortisone for the treatment of bacterial infections and to suppress inflammation, and particularly for treating the ear.

None of the prior art references teach the treatment of difficulty healing wounds or ulcers of the foot and lower extremity of the leg and particularly in diabetic patients.

Diabetic Foot Lesions as a Distinct Medical Entity

Diabetic lesions of the foot ankle and lower extremity of the leg present a unique therapeutic problem in that these lesions are difficult to heal. This fact is reported in the literature.

That healing of diabetic lesions of the foot and lower extremity of the leg present a unique problem is reported in the literature. “Wounds”, Vol. 13, No. 5, September/October 2001, pages 6E-8E, states that wound healing is difficult in people with diabetes. Conferences specifically directed to diabetic foot wound care have taken place. Further evidence that diabetic foot ulcers are unique can be found on pages 32E and 33E of “Wounds”. Diabetic foot ulcers are said to be chronic wounds and exhibit a different biology than what has been classically decribed in the acute healing process. The differences are in neuropathy, structural abnormalities, changes in local regulation of inflammation and changes in blood flow.

Ortho-McNeil-Health Care Provider's Guide to Preventing Diabetes Foot Problems, November 2000, pages 22-26, describes the problem of diabetic foot disease, stating that 15 percent of people with diabetes will experience a foot ulcer and between 14 and 24 percent will require amputation.

Journal of American Podiatric Medical Association, Vol. 92, Number 1, January 2002, pages 1-6, identify the unique problems identified with diabetic foot problems.

Supplement to Podiatry Today, March 2002, How to Treat Lower Extremity Infections, Pollak, pages 9-14, discusses the difficulty associated with selecting the proper anti-infective agent.

Objects of the Invention

An object of this invention is to provide an improved method of treating lesions of the foot, lower extremity of the leg and particularly ulcers of the foot.

A most important object of this invention is for a method which will effectively treat lesions (e.g., ulcers) of the foot and lower extremity of the leg in diabetic patients.

### BRIEF SUMMARY OF THE INVENTION

The herein disclosed invention employs a topical composition containing neomycin, polymyxin and hydrocortisone. Typically, the base for the topical composition will be an ointment or cream base. The cream or ointment containing medication will be applied to the wound or ulcer. The generic composition that the inventor is using contains neomycin and polymyxin B sulfates and hydrocortisone acetate to treat wounds and ulcers on the foot, ankle and lower extremity of the leg.

The preferred topical composition to be used for treating wounds of the foot and particularly diabetic ulcers is a composition containing neomycin, polymyxin B sulfates, zinc bacitracin and hydrocortisone in a topical carrier. A most preferred composition is one in which each gram of the composition contains neomycin sulfate equivalent to 3.5 mg neomycin base, polymyxin B sulfate equivalent to 10,000 polymyxin B units, bacitracin zinc equivalent to 400 bacitracin units, hydrocortisone 10 mg (1%), and white petrolatum, or other suitable topical carrier. In general, the inventor contemplates using compositions containing neomycin sulfate equivalent to 2 to 5 mg neomycin base, polymyxin B sulfate equivalent to 5,000 to 15,000 polymyxin B units and bacitracin zinc equivalent to 200 to 600 bacitracin units and hydrocortisone 5 mgm to 15 mgm (0.5 to 1.5%), in a solid or liquid topical carrier to apply the composition to the wound. The topical carrier comprises at least one of petrolatum, glycerin, propylene glycol and/or water. The optimum composition can be determined by routine experiment by those skilled in the art.

Neomycin sulfate is the sulfate salt of neomycin B and C, which is produced by the growth of Streptomyces fradice Waksman (Fam. Streptomycetaceae). It has a potency equivalent to not less than 600 μg of neomycin standard per mg. calculated on an anhydrous basis.

Polymyxin B sulfate is the sulfate salt of polymyxin B₁ and B₂ which are produced by the growth of Bacillus polymxa (Prazmowski) Migula (Fam. Bacillaceae). It has a potency of not less than 6,000 polymyxin B units per mg. calculated on an anhydrous basis.

Bacitracin zinc is the zinc salt of bacitracin, a mixture of related cyclic polypeptides (mainly bacitracin A) produced by the growth of an organism of the licheniformis group of Bacillus subtilis var Tracy. It has a potency of not less than 40 bacitracin units per mg.

Hydrocortisone. 11β, 17, 21-trihydroxypregn-4-ene-3.20-dione is an anti-inflammatory hormone. Other well known anti-inflammatory steroids such as prednisone and cortisone would be operative in the inventive composition.

Photographs

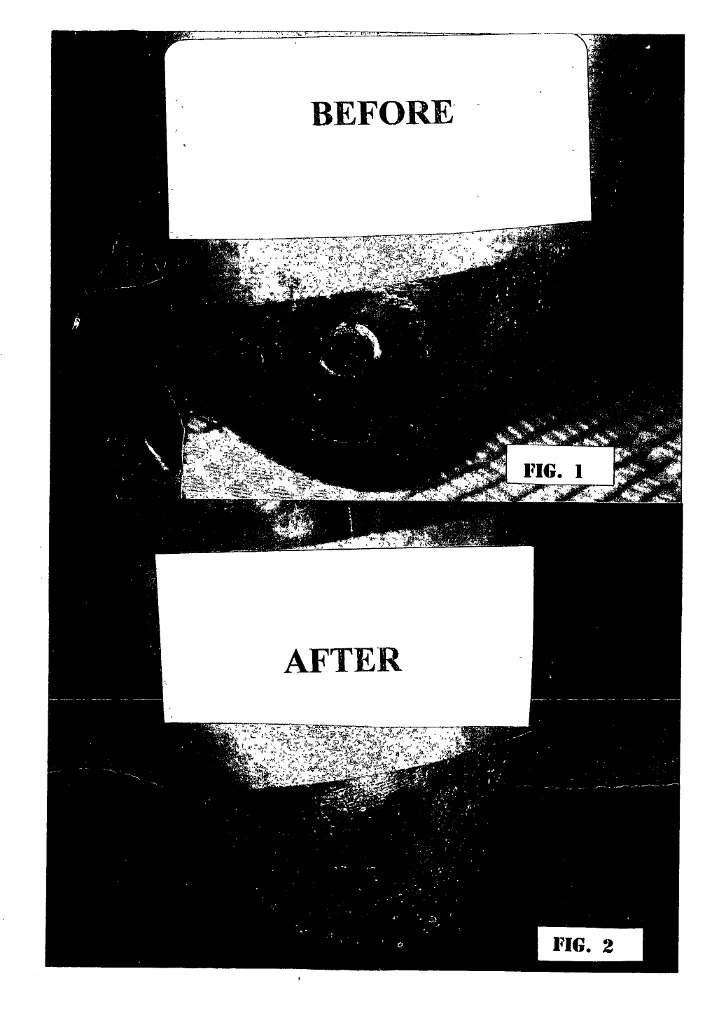

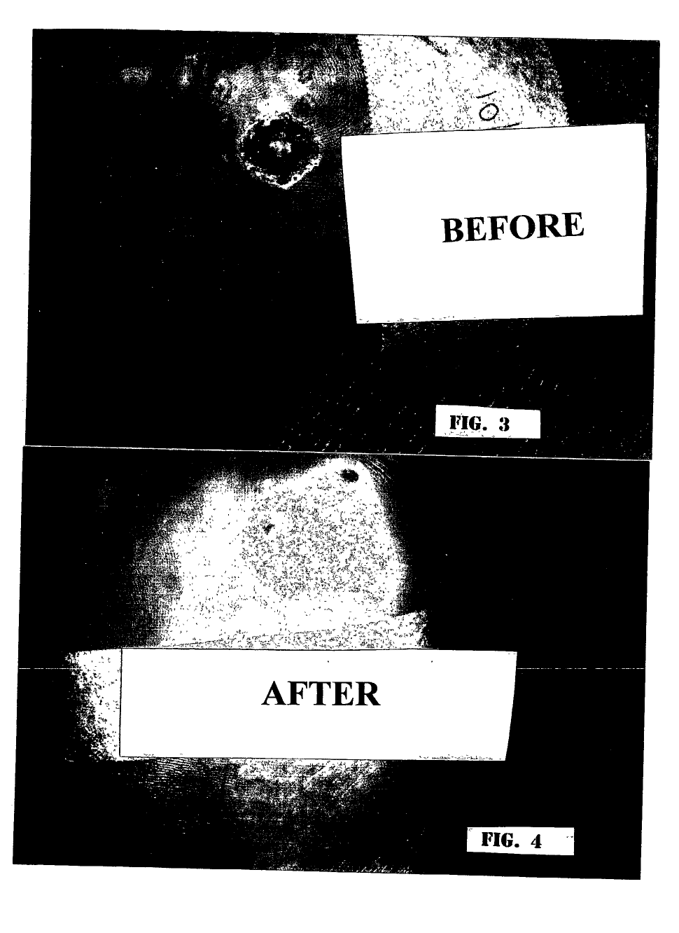

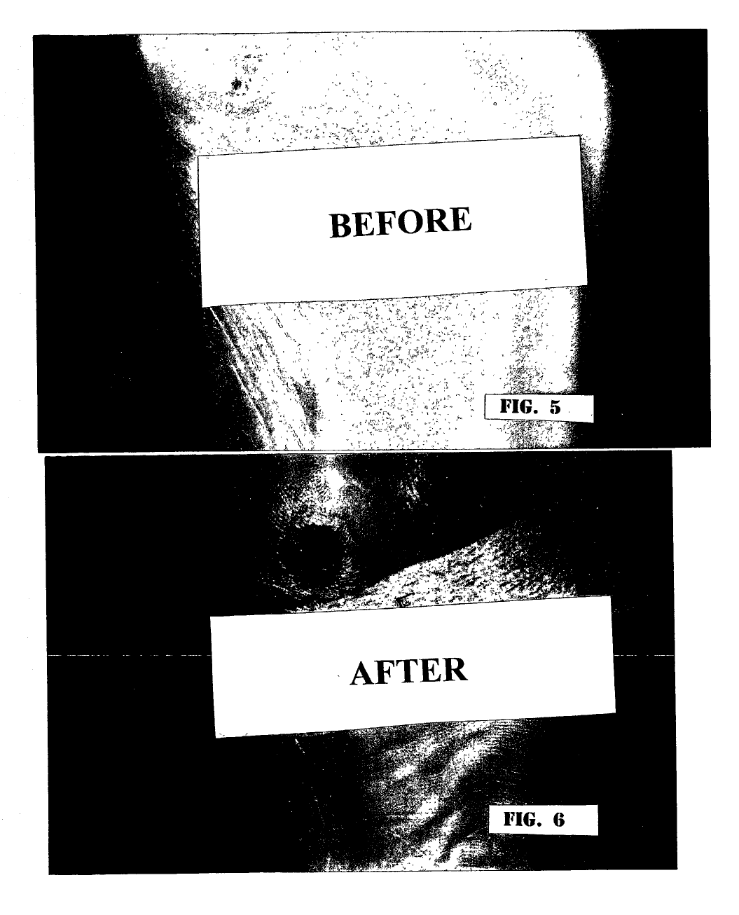

The photographs herein presented, identified as FIGS. 1-6 are representative of the foot ulcers which are being treated by the composition of this invention. Each figure pair shows a before and after treatment.

Patient Treatment Histories

Patient #1.

AB, a 76-year old on nine different medications. Past medical history of hypertension, PVD, heart trouble, arthritis, thyroid problems and high cholesterol. Patient presented to the office with an ulceration on the 2^{nd} right toe to the dermal level which was severely painful. It measured 0.6 cm×0.7 cm. She was given a prescription of the composition of this invention and advised to soak the foot and apply the medication and a bandaid twice per day.

The patient returned to the office five days later stating the pain was completely gone and the ulcer was noted to be 90% healed.

Patient #2.

CD, a 91-year old on eight medications. Past medical history of severe PVD, asthma, and thyroid problems. Patient presented to office with an ulceration on the 2^{nd} left toe to the dermal level measuring 0.4×0.3 cm. She stated that the ulcer was severely painful. She was seen again one month later (as she was unable to arrange transportation sooner). The ulcer was 85% healed and non-painful after using the composition of this invention.

Patient #3.

EF, a 68-year old non-insulin dependent diabetic on six medications. Past medical history of gout, hypertension, diabetes×8-years, and hypercholesterolemia. He presented to the office with an ulceration under the 3^{rd} metatarsal to the dermal level 1.0 cm which has been present for five months. The area is moderately painful. The patient was given a prescription for the composition of this invention and advised how to properly dress the ulcer. One month later the ulcer was 95% healed and non-painful.

Patient #4.

GH, an 83-year old insulin dependent diabetic also on Coumadin for severe PVD. He presents with an ulceration on the 5^{th} toe right foot to the dermal level approximately 1.0 cm. He was given a prescription for the composition of this invention and advised on dressing the ulcer. The toe was noted to be very painful initially. Within several days after using the medication the pain subsided and after approximately two months the ulcer was fully healed.

Patient #5.

IJ, a 68-year old on Coumadin and several other medications for PVD, and heart disease. Past medical history of stroke and open heart surgery. She presents to the office with a deep ulceration on the tip of the right great toe. She was given a prescription for the composition of this invention and advised on local wound care for the ulcer which measured 0.8×0.8 cm. The area was so painful initially that the patient was in tears at the office. After one week the ulcer was 95% healed and completely non-painful. She was broadly smiling at the second visit.

Patient #6.

KL, an 88-year old patient with history of PVD, presents to the office with an ulceration on the top of her left foot measuring 1.2×1.0 cm. The area is swollen and has localized redness with moderate pain. She was given the composition of this invention and advised to dress the wound daily. The ulcer was just below the epidermal level. After two weeks the area was 90% healed and non-painful.

Patient #7.

MN, a 90-year old patient was a non-insulin dependent diabetic, who also suffered from severe edema bilaterally, peripheral vascular disease, and varicosities bilateral.

The patient presented with an ulceration on the right ankle, grade III, 1.2 cm×1.0 cm area, on Oct. 13, 1999. The patient was advised to apply the composition of this invention to the ulceration twice a day and a dry sterile dressing.

She previously had surgery in that area by her general surgeon for an ulceration, which had been non-healing for greater than six months, and had been getting worse over time.

After completing the course of treatment and being seen periodically in the office, she was discharged on Jan. 19, 2000, completely healed.

Patient #8.

OP, a 73-year old female who had been taking multiple medications including prednisone for many years. She suffered from severe peripheral vascular disease and arteriosclerotic vascular disease. She smoked one and one-half packs of cigarettes per day for greater than 50 years. Her skin was very tight, shiny, and thin due to her multiple medical problems.

She had been suffering with a severely painful ulcer on the right ankle on the lateral malleolus for greater than ten months. The ulceration was measured at 1.5 cm×0.8 cm.

She presented with this ulceration on May 16, 2001, and after being advised to apply the composition of this invention and a dry sterile dressing b. i. d., she was followed in the office periodically, and on Jul. 25, 2001, the ulceration was completely healed.

After initially using the medication, the pain subsided dramatically within the first several days.

Patient #9.

QR, a 91-year old female presented to the office with a grade II ulceration on her left great toe, approximately 1 cm in diameter on Oct. 19, 2001.

The patient was on ten different medications with a past history of hypertension, hypercholesterolemia, heart disease, asthma, peripheral vascular disease, and a past surgical history of open-heart surgery.

After utilizing the composition of this invention and weekly visits in the office, the ulceration was noted to be healed on Nov. 12, 2001.

Patient #10.

ST, a 25-year old insulin-dependent diabetic presented with an ulcer on the bottom of his foot approximately 4.5 cm in diameter on Nov. 16, 2001 secondary to a traumatic injury.

The patient was advised to apply the composition of this invention twice a day to the area with a dry sterile dressing. On Dec. 17, 2001, the area was 90% healed.

Patient #11.

UV, a 77-year old, obese, insulin-dependent diabetic presented to the office with an ulceration o the right ankle measured approximately 0.7 cm.

She had a past history of two heart attacks and was on eight different medications.

The ulceration was noted to be a grade II as she presented to the office on Feb. 11, 2002. She was advised to utilize the composition of this invention twice a day with a dressing, and in one month's time the area was 95% healed.

Patient #12.

WX, a 54-year old insulin-dependent diabetic patient who also suffered from peripheral vascular disease and systemic lupus presented with a grade II ulceration of 0.5 cm on Dec. 14, 2001.

After periodic office visits and utilization of the composition of this invention daily, she was completely healed upon her visit of Feb. 1, 2002.

Patient #13.

YZ, an 81-year old patient who was a non-insulin-dependent diabetic, also with heart disease, presented with an ulceration on the fifth right toe, grade II, measured 0.6 cm.

The patient stated that the ulcer was extremely painful. She presented on Oct. 3, 2001 with this ulceration, and on a follow-up visit on Oct. 26, 2001, after utilizing the composition of this invention, the ulceration was 90% healed.

Patient #14.

BC, a patient who presented with an ulcer on the fifth metatarsophalangeal joint on her right foot. The ulceration measured 1.0 cm.

She suffered from severe peripheral vascular disease.

She stated that she had tried to avoid bypass surgery to her legs due to the severity of her medical problems and wanted to try an alternative conservative treatment. It was advised that we would try the composition of this invention to her ulceration.

Initially, she had decreased pain to the ulcer as well as some mild improvement. However, after follow-up visits, the ulceration did not progress as hoped, probably due to the severity of her vascular condition.

She was advised to present back to her vascular surgeon for further workup and probably a bypass on her leg at this time would be advisable.

Patient #15.

DE, a 77-year old female presented with an ulceration on the left ankle treated, for the last ten months, by various doctors at Johns Hopkins Hospital with no improvement. The ulcer measured 0.9 cm×1.1 cm.

She had a past history of peripheral vascular disease, hypertension, and had a triple bypass in 1993.

After utilizing the composition of this invention for one month on a b. i. d. basis, the ulceration was completely healed and non-painful.

Patient #16.

FG, a 68-year old male who was a non-insulin-dependent diabetic also suffering from hypertension presented with an ulceration to the left ankle approximately 10 cm×8 cm with severe edema to the area.

He presented with this problem on Oct. 10, 2001. He was advised to utilize the composition of this invention twice a day as well as elevate the limb to help control the edema.

After several follow-up visits and his home care, he was noted to be completely healed by Nov. 30, 2001.

Patient 17.

HI, an 88-year old female presented with an ulceration to the fifth left toe which was severely painful. She suffered from hypertension, osteoporosis, and thyroid disease. The toe appeared to be contracted, and she was diagnosed with a hammertoe as well.

She presented Nov. 21, 2001 with this problem. The patient preferred a non-surgical approach at this time, and so the composition of this invention was discussed.

However, due to her orthopedic contracture and hammertoe deformity, it was advised that the problem may not heal due to the underlying orthopedic problems. The patient, however, was still insistent that conservative non-surgical treatment be initially tried.

The patient started with the composition of this invention, and after approximately three weeks time with only minimal improvement to the ulcer, the patient decided that surgical treatment to correct the underlying cause of the deformity would be advisable. Thus, the composition of this invention treatment was stopped and surgical consultation was then done at this time.

Advantages from Using the Inventive Composition

The composition of this invention has advantages which other treatment regimens do not possess.

1.) The composition of this invention has been successful where other compositions failed.

2.) The inventive composition healed wounds where other compositions took a long time; a shorter healing time was evident with the inventive composition.

3.) Painful ulcers, not responding to other treatments had pain eliminated or pain level was diminished.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A method for treating difficultly healing wounds or ulcers of the foot, ankle and lower extremity of the foot comprising applying to said wounds or ulcers of the foot, ankle and lower extremity of the foot an effective amount of a topical composition containing a mixture of neomycin, polymyxin and hydrocortisone.

2. A method for treating diabetic wounds or ulcers of the foot and lower extremity of the leg comprising applying to said diabetic wound or ulcer of the foot and lower extremity of the leg an effective amount of a topical composition containing a mixture of neomycin, polymyxin and hydrocortisone.

3. The method for treating difficultly healing wounds or ulcers of claim 1 wherein the topical composition contains neomycin sulfate equivalent to 2 to 5 mg neomycin base, polymyxin B sulfate equivalent to 5,000 to 15,000 polymyxin B units and bacitracin zinc equivalent to 200 to 600 bacitracin units and hydrocortisone 5 mgm to 15 mgm ((0.5 to 1.5%), in a solid or liquid topical carrier.

4. The method for treating difficultly healing wounds or ulcers of claim 3 wherein the topical composition contains neomycin sulfate equivalent to 3.5 mg neomycin base, polymyxin B sulfate equivalent to 10,000 polymyxin B units, bacitracin zinc equivalent to 400 bacitracin units, hydrocortisone 10 mg (1%), and a topical carrier.

5. The method for treating difficultly healing wounds or ulcers of claim 4 wherein the topical carrier comprises at least one of petrolatum, glycerin, propylene glycol and/or water.
